(19) United States
(12) Patent Application Publication
(10) Pub. No.: US 2012/0289994 A1
(43) Pub. Date: Nov. 15, 2012

(54) Occlusion Devices and Related Methods of Use

(75) Inventors: Heather LARSON, Boston, MA (US); Alison Lepordo, Methuen, MA (US)

(73) Assignee: Boston Scientific Scimed, Inc.

(21) Appl. No.: 13/461,115

(22) Filed: May 1, 2012

Related U.S. Application Data
(60) Provisional application No. 61/485,378, filed on May 12, 2011.
(60) Provisional application No. 61/485,735, filed on May 13, 2011.

(51) Int. Cl. A61M 29/00

(52) U.S. Cl. 606/198

(57) ABSTRACT

Embodiments of a medical device and related methods of use are provided in the disclosure. The medical device includes an elongate member having a first end and a second end. The elongate member may also include a plurality of radial projections such that each radial projection extends in a spiral configuration along a length of the elongate member. The medical device may be configured to transition between a compressed delivery configuration and an expanded deployed configuration.

### CROSS-REFERENCE TO RELATED APPLICATIONS

This patent application claims the benefits of priority under 35 U.S.C. §§119-120 to U.S. Provisional Application Nos. 61/485,378 and 61/485,735, filed on May 12 and May 13, 2011, respectively, the entireties of which are incorporated herein by reference.

### FIELD OF THE INVENTION

Embodiments of this disclosure relate generally to medical devices and procedures, and more particularly to medical devices and procedures for occlusion of body parts.

### BACKGROUND OF THE INVENTION

Embolization is a minimally-invasive procedure that involves selective occlusion of blood vessels, by introducing an embolus or other structure to reduce or stop blood flow through the vessel. Embolization of blood vessels can be useful for a variety of medical reasons, including preventing or controlling bleeding due to lesions (organ bleeding, gastrointestinal bleeding, vascular bleeding, and bleeding associated with an aneurysm), or to ablate or otherwise destroy diseased tissue (tumors, vascular malformations, or hemorrhagic processes) by cutting off the blood supply to affected tissue. Further, blood loss during or immediately following surgery may also be prevented by occluding the relevant blood vessels. Embolization of tumors may be performed preoperatively to shrink tumor size, aid in the visualization of a tumor, and minimize or prevent blood loss related to surgical procedures.

One known embolization technique requires implanting occlusive devices into a body lumen, such as an aneurysm, blood vessel, fistula, or other bodily lumens. These implants may serve to reduce the flow of blood through those lumens. One therapy for treating a tumor seeks to occlude a blood vessel upstream of the tumor, thus depriving the tumor of oxygen and nutrients, ultimately shrinking and destroying the tumor.

Conventional embolization techniques limit those procedures to relatively large bodily lumens. Occlusive devices are typically endoluminally delivered by a catheter and advanced to a treatment site, such as a percutaneous entry site, using conventional access procedures. The size of the occlusion device acts as a limiting factor for accessing the treatment site, as larger devices require large-diameter catheters, which may damage body tissues. As a result, known embolization procedures may not be usable in relatively small vessels. While there have been some attempts to provide improved devices and methods for occlusion purposes, these attempts have not been entirely successful.

Thus, a device that ensures effective occlusion of body cavities and facilitates occlusion of body lumens of varying sizes is desirable.

### SUMMARY OF THE INVENTION

Embodiments of the disclosure provide a medical device and its related methods of use. In some embodiments, the medical device may be used for occlusion of blood vessels.

In accordance with an aspect of the present disclosure, a medical device may include an elongate member having a first end and a second end. The elongate member may also include a plurality of radial projections such that each radial projection extends in a spiral configuration along a length of the elongate member. The medical device may be configured to transition between a compressed delivery configuration and an expanded deployed configuration. When in the expanded deployed configuration, the medical device may be configured to retard the flow of fluid through the medical device in a longitudinal direction.

In various embodiments, the medical device may include one or more of the following additional features: the elongate member may include a substantially uniform configuration between the first and second ends; one of the first and second ends of the elongate member may include a longitudinal taper; the medical device may include a mesh having openings sized to retard the flow of blood through the medical device in a longitudinal direction; each of the radial projections may be in a linear configuration when the medical device is in the compressed delivery configuration, and each of the radial projections may transition to the spiral configuration as the medical device transitions to the expanded deployed configuration; the radial projections may unfurl to achieve the spiral configuration; and the medical device may include an additional occlusive agent spaced from and in a non-contacting relationship with the elongate member.

According to another embodiment, a medical device delivery system may include a tubular delivery member defining a lumen and an elongate member at least partially disposed within the lumen. The elongate member may include a first end, a second end, and a plurality of radial projections. Each radial projection may extend in a spiral configuration along a length of the elongate member. The medical device may be configured to transition between a compressed delivery configuration and an expanded deployed configuration. When in the expanded deployed configuration, the medical device may be configured to retard a flow of fluid through the medical device in a longitudinal direction.

In various embodiments, the medical device delivery system may include one or more of the following additional features: the delivery system may include a pusher wire for advancing the elongate member out of the tubular delivery member; one of the first and second ends may include a longitudinal taper; the medical device may include one of a mesh and a foam configured to retard a flow of blood through the medical device in a longitudinal direction; each of the radial projections may be in a linear configuration when the medical device is in the compressed delivery configuration, and each of the radial projections may transition to the spiral configuration as the medical device transitions to the expanded deployed configuration; the radial projections may unfurl to achieve the spiral configuration; and the medical device may include an additional occlusive agent spaced from and in a non-contacting relationship with the elongate member.

A further aspect of the present disclosure may include a method of occluding a blood vessel of a patient. The method may include advancing a catheter including a medical device to a position within the blood vessel. The medical device may include a first end, a second end, and a plurality of radial projections. Each radial projection may extend in a spiral configuration along a length of the elongate member. The medical device may be configured to transition between a compressed delivery configuration and an expanded deployed configuration. When in the expanded deployed configuration, the medical device may be configured to retard a flow of fluid through the medical device in a longitudinal direction. The method may further include advancing the medical device out of a lumen of the catheter. The advancing step may result in expansion of the medical device from the compressed delivery configuration. The device may be configured to expand until at least one of the radial projections engages a wall of the blood vessel.

In various embodiments, the method may include one or more of the following additional features: advancing the medical device out of the lumen of the catheter may include manipulating a proximal end of a pusher mechanism; the method may further include delivering a plurality of discrete occlusive agents to a location adjacent the location of the medical device; each radial projection may be in a linear configuration when the medical device is in the compressed delivery configuration, and each of the radial projections may transition to the spiral configuration as the medical device transitions to the expanded deployed configuration; the radial projections may unfurl to achieve the spiral configuration; and the medical device may include a mesh having openings sized to retard the flow of blood through the medical device in a longitudinal direction.

It is to be understood that both the foregoing general description and the following detailed description are exemplary and explanatory only and are not restrictive of the disclosure, as claimed.

### BRIEF DESCRIPTION OF THE DRAWINGS

The accompanying drawings, which are incorporated in and constitute a part of this specification, illustrate embodiments of the disclosure and together with the description, serve to explain the principles of the disclosure.

FIG. 1 is a schematic view of a medical device according to an embodiment of the present disclosure.

FIG. 2 is a schematic view of an alternate medical device having tapered ends according to an embodiment of the present disclosure.

FIG. 3 is a schematic view of a cross-sectional view of the medical device along the plane A-A′ (FIG. 1).

FIG. 4 is a schematic view of the medical device of FIG. 3, depicting compressed and expanded configurations.

FIG. 5 is a schematic view of another embodiment of the medical device having a twist along the length of the device.

FIGS. 6A, 6B, and 6C are schematic views of the medical device of FIG. 1 inserted into a catheter, the medical device advancing from the distal end of the catheter, and the medical device being deployed in a blood vessel, respectively.

FIG. 7 is a schematic view of the medical device deployed in the blood vessel along with additional occlusive agents.

### DESCRIPTION OF THE EMBODIMENTS

Reference will now be made in detail to embodiments of the disclosure, an example of which is illustrated in the accompanying drawings. Wherever possible, the same reference numbers will be used throughout the drawings to refer to the same or like parts.

### Overview

The present disclosure generally relates to occlusion devices such as a vascular occlusion device that may assist in occluding a body lumen such that the fluid communication between the sides partitioned by the occlusion device is reduced. In particular, an embodiment of the disclosed occlusion device may include an elongate body having a retention mechanism for securing the device within the body lumen. In general, the retention mechanism may include a number of retention members, which may be protrusions that contact the inner surface of the body lumen to prevent fluid flow. In addition, the occlusion device may include a configuration, such as a mesh-based plug, that assumes a compressed state where the retention members are collapsed with the elongate body, and an expanded state where the retention members expand, in a radial direction, for example. The occlusion device may advance to a predetermined location in the compressed configuration, and, upon deployment, the occlusion device may transform to the expanded configuration. In the expanded state, the device may conform to the contour of the body lumen such that the lumen may be substantially blocked.

The occlusion device may facilitate occlusion of blood vessels or other body lumens, including, for example, portions of the gastrointestinal tract and lumens in the pulmonary, biliary, urinary, and neural systems. In embodiments within blood vessels, the expanded device may provide for a large surface area to promote blood clotting. Further, the compressed and expanded configurations allow insertion of the device to a blood vessel having substantially smaller diameter. The embodiments of the present disclosure are discussed in relation to embolization. It should be understood, however, that such occlusion devices and methods are not limited to embolization of the blood vessels and may be applicable to any body lumen that could benefit from the use of the occlusion device.

### Exemplary Embodiments

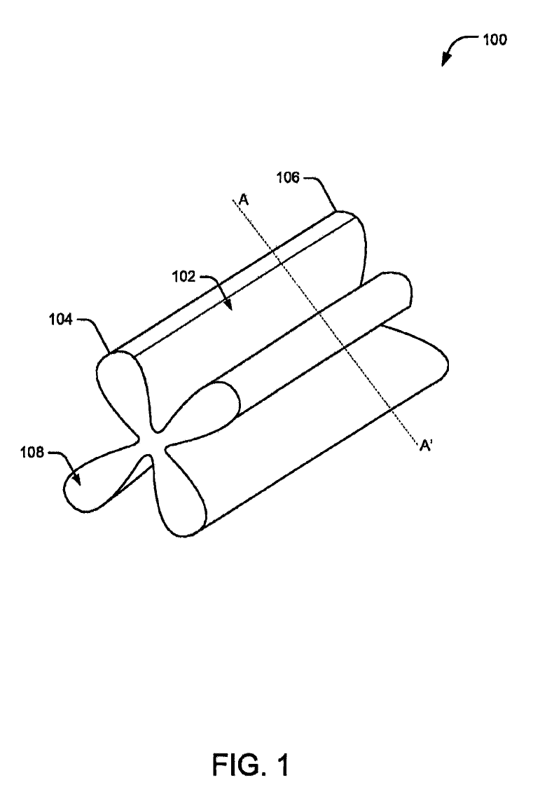

FIG. 1 illustrates an exemplary embodiment of a medical device 100 that may be implanted in patient's body for occlusion purposes. The medical device 100 may include a generally elongate member 102 having a first end 104 and second end 106. In addition, a number of radial projections, hereafter retention members, 108 may protrude from an outer surface of the elongate member 102. The medical device 100 may be an expandable mesh-based device that securely anchors itself within a body lumen as a plug to block the flow of fluid across or through the device. In addition, those of ordinary skill in the art will recognize that device 100 may have any suitable construction and/or configuration. For example, the device 100 may be made from a foam-like or fibrous material that is expandable.

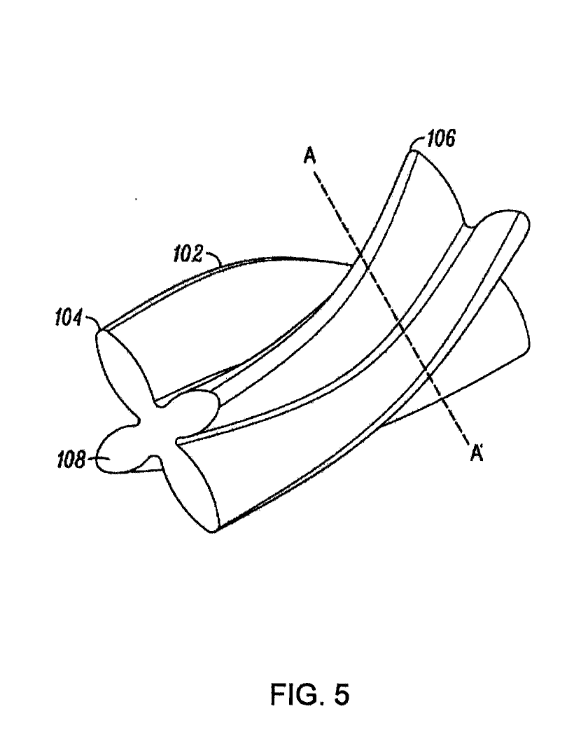

The member 102 may be an elongate structure having retention members 108 protruding as spokes from the member 102. As shown, the medical device 100 forms a flower-like configuration with retention members 108 being the “petals” of this flower-like configuration. Each retention member 108 protrudes from the member 102 in a radial direction, and extends along the length of the member 102 from the first end 104 to the second end 106, as discussed in greater detail below. In addition, the medical device 100 may assume two configurations—compressed and expanded. The medical device 100 may be compressed to reduce the overall diameter, and this capability allows insertion to relatively smaller body lumens, such as, for example, those in a patient's brain. In the expanded configuration, the medical device 100 may expand in the radial direction. Not only the radius of the elongate member 102 increases, but each retention member 108 expands such that the cross-sectional configuration of the medical device 100 expands, for example into a circular-like structure, having a diameter generally greater than the diameter of the body lumen. This expanded configuration provides a self-securing mechanism to anchor the device 100 and block fluid flow through the body lumen. The two configurations will be discussed in detail in the following section in connection with FIG. 4.

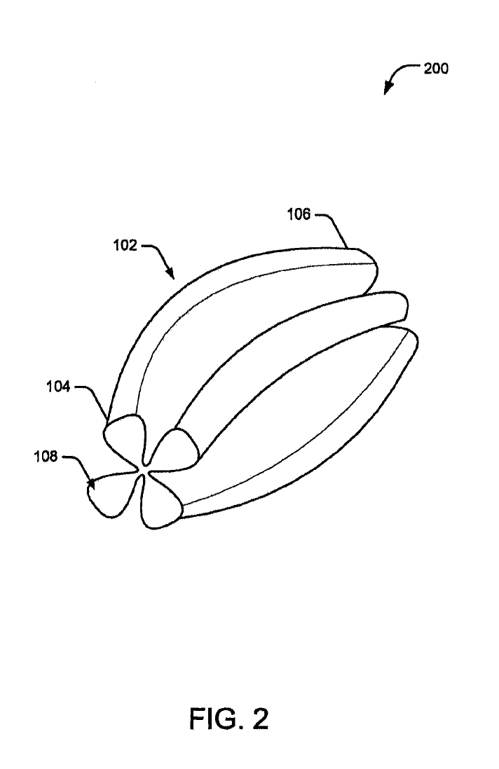

The elongate member 102 may include a cylindrical or cylinder-like configuration having a substantially symmetrical cross-section along its length. Alternatively, member 102 may have a varying cross-sectional configuration between the first and second ends 104, 106. In addition, elongate member 102 may have a tapered configuration such that one of the first end 104 or second end 106 may include a smaller dimension relative to other portions of the elongate member. FIG. 2 illustrates an embodiment of the device 100 having tapered first and second ends 104, 106. In another embodiment, the cross-sectional configuration of the member 102 may vary along its length. It should be understood that the elongate member 102 may assume a funnel, a spherical, spheroid, or egg shaped configuration.

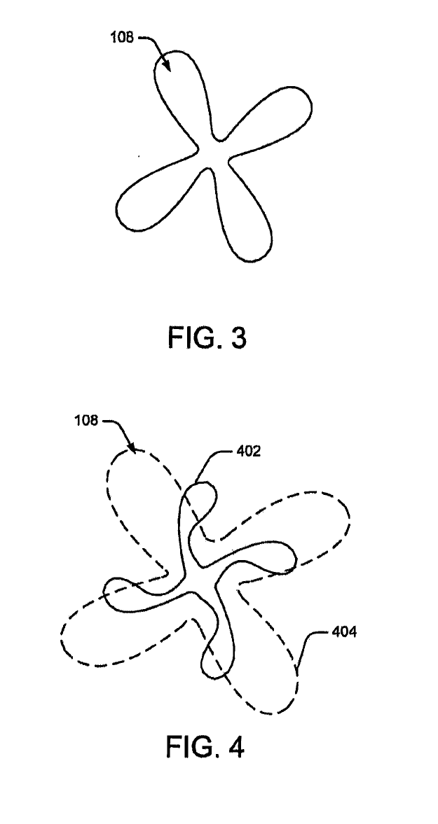

FIG. 3 illustrates a cross-sectional view of the medical device 100 (taken along plane A-A′ in FIG. 1) that represents a petal-like configuration. Alternatively, the medical device 100 may include a four-lobed device; each retention member 108 resembling a lobe-like structure. In general, retention members 108 may include circular, oval, or an elliptical shape. As shown, four retention members 108 protrude symmetrically from the outer surface of the member 102, and each retention member 108 may assume a geometrical configuration similar to the flower petal to form a flower-like shape of the medical device 100. Each retention member 108 comes into contact with and engages a body lumen wall.

Although the illustrated embodiment depicts four retention members 108 protruding symmetrically around the member 102, the number of retention members 108 surrounding the elongate member 102 may vary. In an embodiment, three retention members 108 may be disposed around the member 102 at about 120 degrees to each other. Alternatively, the medical device 100 may include any suitable number of retention members 108, which may be disposed around the member 102 at any suitable angle. Those skilled in the art will understand that retention members 108 may have varying diameters and/or lengths such that no two retention members may be substantially similar. The diameter and length of each retention member may be varied to account for placement in a body lumen having a particular configuration, such as an elbow-bend portion of a lumen. In another embodiment, at least three retention members 108 may contact the body lumen. The adjacent retention members 108 may substantially contact each other or there may be no point of contact between them. Furthermore, retention members 108 either may include a smooth outer surface or may include a rough outer surface.

The retention members 108 extend along the length of the member 102, as shown in FIG. 1. As shown, each retention member 108 extends along the length of the elongate member 102 from the first end 104 to the second end 106. Thus, each retention member 108 may be an elongate tubular structure having a petal-like cross-sectional shape. Alternatively, retention members 108 may not run completely along the length of member 102. Additionally, some of the members 108 may extend along the length, and others may only extend for a portion of the length from one of the first end 104 or second end 106.

Furthermore, one or both of the first and second ends 104, 106 may be designed to reduce trauma and irritation to surrounding tissues. The ends 104, 106 may include rounded or beveled terminal ends and/or faces, for example.

As discussed, the medical device 100 may assume a compressed configuration for inserting the device within a target body lumen and once deployed, the device 100 may expand into its expanded configuration to mitigate migration. FIG. 4 is an exemplary cross-sectional view of the medical device 100 illustrating a collapsed state (depicted by a solid line 402) and an expanded state (depicted by a dotted line 404). As shown, the medical device 100 may expand from the compressed state to the expanded state in the radial direction. The diameter or other cross-sectional dimension of the medical device 100 may increase as the device expands. Furthermore, the diameter or other cross-sectional dimension of each retention member 108 may also increase. In some embodiments, retention members 108 may also diverge or unfurl from one another as the device 100 expands. The compressed configuration allows insertion of the device 100 to body lumen having a substantially small diameter. In the expanded state, the retention members 108 may expand in diameter such that the overall diameter of the device 100 becomes greater than the diameter of the body lumen. This radial expansion of the device 100 allows the device to frictionally retain itself at the deployed location. In addition, the expanded configuration securely fitted within the body lumen may also block the flow of bodily fluids, such as blood, across the device 100.

In an embodiment, each retention member 108 may exhibit a substantial linear uniform configuration when the medical device is in the compressed delivery configuration. As the medical device 100 transitions to the expanded deployment configuration, each retention member 108 transitions to a varying configuration such as a spiral twist along the length of the medical device 100. Such a configuration is discussed in greater detail in relation to FIG. 5 in the following sections.

To facilitate configuration change, the medical device 100 may be a self-expandable device that expands from its compressed configuration once positioned within the body. Alternatively, the device 100 may be made of a material that can be compressed by a suitable restraint and expanded when the restraint is removed. In such embodiments, the medical device 100 may be made from a shape memory material (such as Nitinol or other suitable metals or metal alloys) or an elastic and/or flexible material such as synthetic fiber, rubber, polymer, foam, or other known materials.

In another embodiment, the medical device 100 may be connected to an expandable member (not shown) such as a balloon that may be manually expanded by pumping air, saline, or other suitable inflation fluids into the balloon. The expandable member may be connected to one or both of the elongate member 102 and retention members 108 in order to expand multiple portions of the device 100. In some embodiments, the expandable member may selectively control the expanded size of the device 100. The capabilities provided by the expandable member enables one particular medical device 100 to fit into and occlude body lumens of varying dimensions, reducing the need for selecting a particularly sized device 100 for a particular target lumen. The expandable member or self-expandable characteristics may ensure that the expanded configuration of the device 100 is larger than the body opening where device 100 may be positioned.

Apart from the self-securing mechanism provided by the expansion of the device 100, the outer surface of the medical device 100 may include geometrical structures (not shown) that may act as additional securing mechanisms. These geometrical structures may include hooks, barbs, or spikes that may assist in engaging the medical device 100 with the surrounding tissues. The geometrical structures may be deployed at one of the first end 104 or second end 106. Alternatively, these structures may be positioned along the length of the device 100.

The medical device 100 may be made of a mesh-based structure having a number of wires crossing over each other to create openings or cells throughout the structure. The mesh structure may have substantially small openings. These openings of the mesh may accomplish embolization of blood as the blood flows across the medical device 100. For example, the mesh may include openings sized to retard the flow of blood through the medical device 100 in a longitudinal direction. In other embodiments, the medical device 100 may be a solid structure, biocompatible foam, or biocompatible fibrous structure. In such embodiments, the device 100 may be coated or covered as generally known in the art. The coating or covering may include any synthetic, fibrous, or biocompatible material that may be compatible with living tissues. It should be readily apparent to those of ordinary skill in the art that the device 100 may be either partially or completely coated with any suitable lubricous material, antibiotic coating, or other required material.

The medical device 100 renders capabilities of occlusion within a body lumen where it is deployed. The medical device 100 blocks the body lumen that results in slowing down the flow of body fluid across the device, and ultimately stops the fluid flow. The expanded configuration of the medical device 100 may also assist in occlusion as the expanded device secures itself within the body lumen limiting the fluid to flow across the device 100. For example, the mesh-based device 100 once deployed within a blood vessel, expands itself and engages the surrounding lumen walls to substantially block the vessel. Initially, the blood may flow through the openings of the mesh; over time, however, the blood may start to clot around the mesh, as the openings are substantially small in size, eventually blocking the flow of blood across the device 100.

In some embodiments, the medical device 100 may include additional occlusive agents added to at least a portion of the medical device 100. Occlusive agents may be devices that act as additional barriers to the flow of fluid through the device 100, such as spherical or coil shaped particles, fabric, glues, or other known devices that may reduce the occlusion time. These agents may be disposed at one of the first end 104 or second end 106 of the medical device 100. Alternatively, the occlusive agent may be disposed on any portion of the outer surface of the medical device 100, or within medical device 100. In some embodiments, the additional occlusive agents may not be connected to the medical device 100, and positioned adjacent to one end of the device 100.

In addition, the medical device 100 may include structural modifications that may facilitate faster occlusion. FIG. 5 illustrates an alternate embodiment of the medical device 100 having a varying cross-section. As shown, the elongate member 102 may include a spiral twist, which may assist in occlusion. The spiral twist may create turbulent flow of blood around the device 100 that may decrease the occlusion time. In some embodiments, each retention member 108 may exhibit a linear configuration (as shown in FIG. 1) when the medical device 100 is in the compressed delivery configuration, and in the expanded deployment configuration, each member 108 may transition to the spiral configuration. Although, the illustrated embodiment depicts only one spiral twist, it should be understood that the member 102 may include multiple twists.

Medical device 100 may be a mesh-based structure made of any suitable material that is compatible with living tissue or a living system, non-toxic or non-injurious, and does not cause immunological reaction or rejection. Such materials may include nitinol, PET, ePTFE, fabric, and suitable nickel and titanium alloys. In some embodiments, the medical device 100 may include biodegradable and/or bioresorbable characteristics. The device 100 may decompose or degenerate into a water-soluble substance, which dissolves or erodes over time upon exposure to a body fluid. In other implementations, the medical device 100 material may not be soluble, but it may degrade into sufficiently sized particles that may be naturally passed through the body. For example, once occlusion is no longer required, the medical device 100 may be eliminated from the body by excretion or metabolized by the body. Furthermore, one or both of the elongate member 102 and retention members 108 may be biodegradable or resorbable.

In addition, a portion of the medical device 100 may include a coating. In one embodiment of the present disclosure, the medical device 100 may be coated with an anti-bacterial material to inhibit bacterial growth on the surface of the medical device 100. The antibiotic coating may contain an inorganic antibiotic agent disposed in a polymeric matrix, which may adhere the antibiotic agent to the medical device surface. In another embodiment of the present disclosure, the medical device 100 may be coated with a lubricious coating to facilitate convenient insertion in the body tissues, such as blood vessels. The lubricious coating on the medical device 100 provides a low friction surface to the tissues. In addition, the coating or covering, such as lubricious coating, may prevent tissue in-growth. Alternatively, the coating and/or covering may be made of suitable material that promotes tissue in-growth, which may support permanent deployment of the device 100.

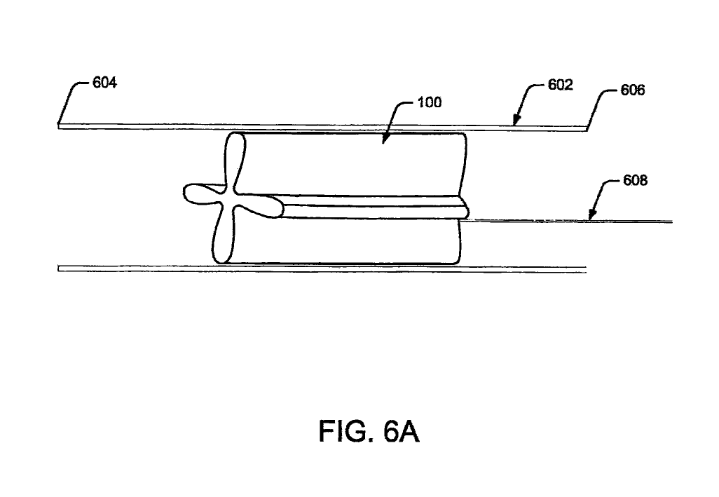

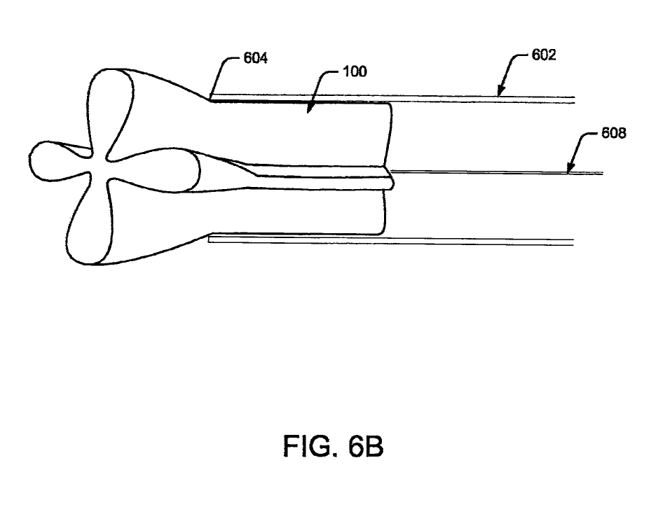

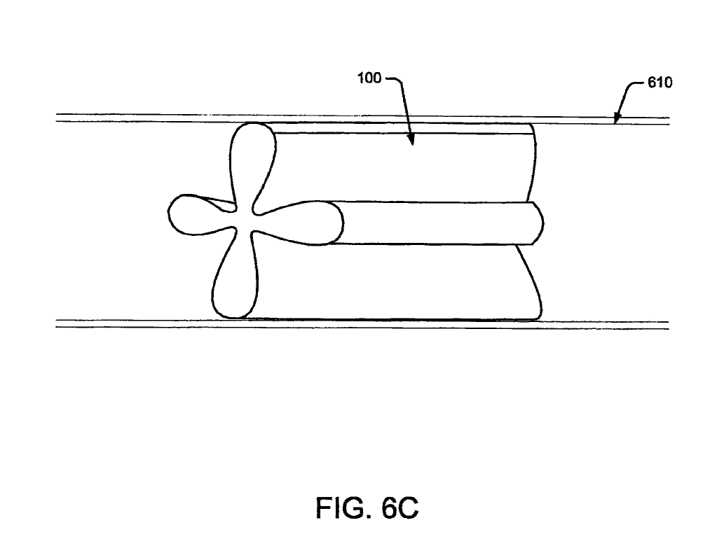

FIGS. 6A-C illustrate embodiments of the present disclosure including a method of using the medical device 100 to facilitate occlusion within a patient's body, at locations such as blood vessels. In such embodiments, the medical device 100 may be used to block the flow of blood across the medical device 100. Those skilled in the art will understand that the present disclosure may be implemented for occlusion in any desired location within the body such as an aneurysm, blood vessel, fistula, or other bodily lumens.

A catheter 602, such as a micro-catheter, may be employed to facilitate insertion of the medical device 100 to the desired location. As shown, the catheter 602 may be an elongate tube that may contain at least a portion of the medical device 100. The catheter may be a highly flexible tube with a relatively small outer diameter in the range of 0.5 mm to 2 mm, or 1-5 F. The micro-catheter having such small diameters may be inserted into any body lumen, including blood vessels in a patient's brain. The catheter 602 may include a distal end 604 and a proximal end 606. In some embodiments, the catheter 602 may include one or more channels, and the device 100 may be inserted through one of these channels. Furthermore, the outer surface of the catheter 602 may include a coating such as a lubricious coating that reduces the friction imposed on the surrounding tissues during insertion or retraction.

The catheter 602 may advance into the body tissues through a natural body opening or through a surgically created opening known to those skilled in the art. The catheter 602 advances through the body tissue until it reaches the desired location such as the portion of the blood vessel that needs to be blocked. Those skilled in the art will understand that catheter 602 may also have one or more mechanisms that facilitate steering the distal end 604, to aid in appropriate medical device delivery.

Medical device 100, outlined above, having an expanded and a compressed configuration, allows the device 100 to be passed through the micro-catheter 602. The medical device 100 can be collapsed into its compressed configuration and inserted into the lumen of the catheter 602. This configuration may be achieved by, for example, applying tension along the radial axis of the device 100. The compressed configuration of the device 100 may be of any shape suitable for easy passage through the lumen of the catheter 602. FIG. 6A illustrates the device 100 in a compressed state advancing through the catheter 602.

Once the medical device 100 is collapsed and inserted into the catheter 602, it may be urged along the lumen of the catheter 602 toward the distal end 604. This may be accomplished by using a pusher wire 608 or the like to abut against and push the device 100 so that it may be advanced along the catheter 602. Advancing the medical device 100 out of the lumen of the catheter 602 may include manipulating (for example, pushing) a proximal end of the pusher wire 608. When the device 100 begins to exit the distal end 604, which is positioned adjacent the desired deployment location, the device 100 may self-expand to an expanded configuration. The expanded configuration may be defined by the shape of the medical device 100 when it expands to generally conform to the contour of the body lumen. The expanded configuration may be either preset or controlled by the blood lumen where the device 100 will be deployed. In addition, the elongate member 102 may transition to a spiral configuration once it expands from the compressed configuration.

FIG. 6B illustrates an embodiment of the distal end of the catheter 602 depicting the medical device 100 expanding from the compressed configuration. Superelastic alloys, such as nitinol, may be useful in this application because of their ability to return to a particular configuration after being elastically deformed to a collapsed state. Furthermore, the elastic or self-expandable characteristics of the medical device material may assist in the expansion of the device from its compressed configuration. As shown, urging the medical device 100 out of the distal end of the catheter 602 tends to expand the device 100 resiliently. Alternatively, a suitable expansion mechanism (not shown) may be used to facilitate expansion of medical device 100 after it is delivered to a desired location.

Although the device 100 may resiliently return to its initial expanded configuration (shape prior to being compressed for passage through the catheter 602), it should be understood that the device 100 may not always return entirely to that shape. The expanded configuration of the medical device 100 may include a diameter larger than the inner diameter of a blood vessel in which it is deployed. In such a situation, the blood vessel will prevent the device 100 from completely returning to its expanded configuration.

Once the medical device 100 completely exits the distal end of the catheter 602, the device expands, and positions itself at the desired location such as the desired location within the blood vessel. Subsequently, catheter 602 may be retracted from the body. In some embodiments, the device 100 may be erroneously positioned. Thus, it may necessary to compress and withdraw the device 100 into catheter 602. In such embodiments, elongate member 102 may be provided with a suitable mechanism for re-compressing elongate member 102. For example, a proximal end of elongate member 102 may include a plurality of rings (not shown) disposed radially about the periphery of the proximal end. A cinching member (not shown), such as, for example, a suture, may be threaded through the plurality of rings. In addition, a distal end of pusher wire 608 may include a hook for grasping and proximally withdrawing the cinching member, thereby radially compressing elongate member 102.

FIG. 6C illustrates an embodiment of the medical device 100 having an expanded configuration, and deployed within a blood vessel 610. As shown, the medical device 100 anchors itself within the blood vessel 610 such that the retention members 108 form a seal with the surrounding vessel tissues. The expansion may secure the device 100 to the nearby tissue as the expanded configuration includes a diameter generally greater than the diameter of the vessel 610. The spring force of the device 100 pushes the device 100 against the inner wall of the vessel 610, and this force may retain the device 100 at the deployed position, minimizing migration. In some embodiments, the above-described additional securing mechanisms (barbs, hooks, or spikes) come into contact and engage surrounding tissue. In some embodiments, these additional securing mechanisms may penetrate a portion of the lumen wall.

Furthermore, radiopaque or sonoreflective markings (not shown) may be added to an exterior surface of the medical device 100. These markings facilitate detection of a position and/or orientation of the medical device 100 within the patient's body. A surgeon, with the aid of suitable imaging equipment, may view these markings to enable optimal positioning of the medical device 100 and to avoid potential damage to sensitive blood vessels. To this end, the pusher wire 608 may help in maneuvering the medical device 100 to the desired location.

Once deployed within the blood vessel 610, the medical device 100 facilitates blood clotting or blockage. The mesh-based structure of the device 100 assists in clotting the blood. In addition, the spiral twist along the length of the device 100, shown in FIG. 4, may increase the occlusive properties of the device. In general, the mesh includes cells or openings, which are sized such that the device 100 has a low porosity. After deployment, initially blood may flow through and around the device 100. As the mesh includes openings or cells with fine openings, the device 100 retards the flow of blood in a longitudinal direction and subsequently, blood cells may start clotting around these openings. Moreover, the spiral twist may induce a thrombogenic effect as the twist creates turbulent flow around the device 100 that may decrease the occlusion time. Thus, the medical device 100 promotes blood occlusion.

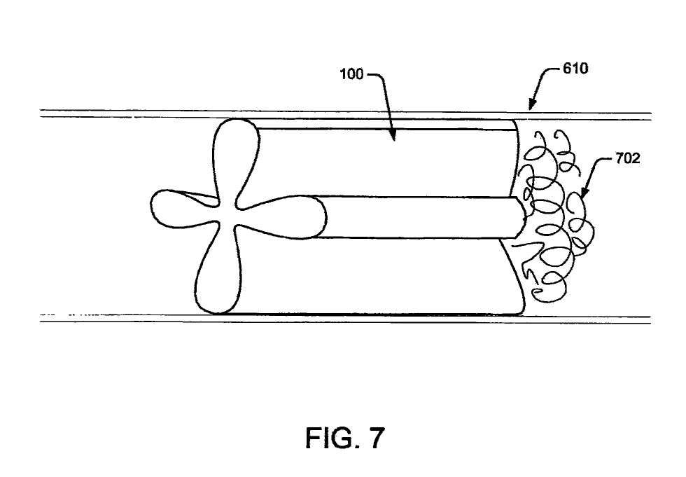

In some embodiments, additional occlusive agents may be added to reduce the occlusion time. FIG. 7 is an embodiment of the medical device 100 disposed within the blood vessel 610 along with additional occlusive agents 702. In one embodiment, one the medical device is deployed, a plurality of discrete occlusive agents 702 may be introduced to a location adjacent the location of the medical device 100. The occlusive agents 702 may be introduced using the pusher wire 608, catheter 602, or other known mechanisms. Alternatively, the occlusive agent 702 may be added to the material for manufacturing the medical device 100 or the occlusive agent may be connected to the outer surface of the medical device 100. As shown, occlusive agents may include spherical or coiled particles, which are compatible with the body tissues. Alternatively, any suitable occlusive agent including fiber, glues, or similar devices known to those skilled in the art may be employed. These agents block the flow of blood across the device 100. Although the illustrated embodiment depicts the occlusive agents 702 disposed at the second end 106, those skilled in the art will understand that the occlusive agents 702 may be disposed at any desired position surrounding the device 100 such that the occlusion time may be reduced. In addition, the additional occlusive agents 702 may be disposed within or upon the medical device 100. Furthermore, the occlusive agents 702 may be spaced from and in a non-contacting relationship with the elongate member 102.

In addition, the medical device 100 may be deployed permanently or temporarily. If the device 100 is required to permanently occlude a channel in the patient's body, such as the vessel 610, the catheter 602 may be retracted from the body leaving the device deployed within the patient's body. To this end, the medical device 100 may be made of any suitable biocompatible material that may impose minimum risk of damaging the surrounding tissues. The mesh structure of the device 100 may allow for tissue in growth for permanent fixation of the device.

Alternatively, the medical device 100 may remain within the body for a predetermined period of time and subsequently, the device 100 may be removed. A catheter, such as the micro-catheter 602, or similar devices may be used to retract the device out of the body. Alternatively, the medical device 100, being bioresobable, starts resorbing over time. The resorption nature of the medical device 100 avoids the step of manually removing the medical device 100.

The present disclosure provides a medical device that facilitates occlusion of blood flow within a blood vessel. Further, the compressed configuration of the device allows insertion of the device within the body through a micro-catheter. Once deployed the device expands and provides occlusion within a short span of time.

Embodiments of the disclosure may be used in any medical or non-medical procedure, including any medical procedure where occlusion of a body lumen is desired. In addition, at least certain aspects of the aforementioned embodiments may be combined with other aspects of the embodiments, or removed, without departing from the scope of the invention.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A medical device comprising:
an elongate member having a first end and a second end, the elongate member including a plurality of radial projections, each of the radial projections extending in a spiral configuration along a length of the elongate member,
wherein the medical device is configured to transition between a compressed delivery configuration and an expanded deployed configuration, and wherein, when in the expanded deployed configuration, the medical device is configured to retard the flow of fluid through the medical device in a longitudinal direction.

2. The medical device of claim 1, wherein the elongate member includes a substantially uniform configuration between the first and second ends.

3. The medical device of claim 1, wherein one of the first and second ends includes a longitudinal taper.

4. The medical device of claim 1, wherein the medical device includes a mesh having openings sized to retard the flow of blood through the medical device in a longitudinal direction.

5. The medical device of claim 1, wherein each of the radial projections is in a linear configuration when the medical device is in the compressed delivery configuration, and wherein each of the radial projections is configured to transition to the spiral configuration as the medical device transitions to the expanded deployed configuration.

6. The medical device of claim 5, wherein the radial projections unfurl to achieve the spiral configuration.

7. The medical device of claim 1, wherein the device includes an additional occlusive agent spaced from and in a non-contacting relationship with the elongate member.

8. A medical device delivery system, comprising:
a tubular delivery member defining a lumen; and
an elongate member at least partially disposed within the lumen, wherein the elongate member includes a first end and a second end, the elongate member further including a plurality of radial projections, each of the radial projections extending in a spiral configuration along a length of the elongate member,
wherein the medical device is configured to transition between a compressed delivery configuration and an expanded deployed configuration, and wherein, when in the expanded deployed configuration, the medical device is configured to retard a flow of fluid through the medical device in a longitudinal direction.

9. The system of claim 8, further comprising a pusher wire for advancing the elongate member out of the tubular delivery member.

10. The system of claim 8, wherein one of the first and second ends includes a longitudinal taper.

11. The system of claim 8, wherein the medical device includes one of a mesh and a foam configured to retard a flow of blood through the medical device in a longitudinal direction.

12. The system of claim 8, wherein each of the radial projections is in a linear configuration when the medical device is in the compressed delivery configuration, and wherein each of the radial projections is configured to transition to the spiral configuration as the medical device transitions to the expanded deployed configuration.

13. The system of claim 12, wherein the radial projections unfurl to achieve the spiral configuration.

14. The system of claim 8, wherein the device includes an additional occlusive agent spaced from and in a non-contacting relationship with the elongate member.

15. A method of occluding a blood vessel of a patient, the method comprising:
advancing a catheter including a medical device to a position within the blood vessel, wherein the medical device comprises:
an elongate member including a first end and a second end, the elongate member further including a plurality of radial projections, each of the radial projections extending in a spiral configuration along a length of the elongate member,
wherein the medical device is configured to transition between a compressed delivery configuration and an expanded deployed configuration, and wherein, when in the expanded deployed configuration, the medical device is configured to retard a flow of fluid through the medical device in a longitudinal direction; and
advancing the medical device out of a lumen of the catheter, wherein advancing the medical device results in expansion of the medical device from the compressed delivery configuration, wherein the medical device is configured to expand until at least one of the radial projections engages a wall of the blood vessel.

16. The method of claim 15, wherein advancing the medical device out of the lumen of the catheter includes manipulating a proximal end of a pusher mechanism.

17. The method of claim 15, wherein the method further includes delivering a plurality of discrete occlusive agents to a location adjacent the location of the medical device.

18. The method of claim 15, wherein each of the radial projections is in a linear configuration when the medical device is in the compressed delivery configuration, and wherein each of the radial projections is configured to transition to the spiral configuration as the medical device transitions to the expanded deployed configuration.

19. The method of claim 18, wherein the radial projections unfurl to achieve the spiral configuration.

20. The method of claim 15, wherein the medical device includes a mesh having openings sized to retard the flow of blood through the medical device in a longitudinal direction.
